(19) United States
(12) Patent Application Publication
(10) Pub. No.: US 2012/0316446 A1
(43) Pub. Date: Dec. 13, 2012

(54) RAMAN SPECTRAL ANALYSIS FOR DISEASE DETECTION AND MONITORING

(75) Inventor: Sinisa Vukelic, New York, NY (US)

(73) Assignee: Sinisa Vukelic, New York, NY (US)

(21) Appl. No.: 13/490,802

(22) Filed: Jun. 7, 2012

Related U.S. Application Data
(60) Provisional application No. 61/520,271, filed on Jun. 7, 2011.

(51) Int. Cl. A61B 6/00; G01J 3/44

(52) U.S. Cl. 600/476; 356/301

(57) ABSTRACT

A system and method for Raman spectral analysis for diagnostic purposes to identify the disease state of biological tissue. The sampled tissue can be any human or animal tissue, including but not limited to prostate, breast, cervical tissue, etc. Raman spectroscopy is used to provide Raman bands which are decomposed and their chemical fingerprints identified after mathematical modeling. The method enables identification of chemical compounds in the tissue being diagnosed. Comparison with reference samples permits inferences concerning the presence or absence of disease, the stage of disease, and/or the response of disease to treatment. The method provides sufficient information about the chemical composition of the tissue to differentiate between normal and abnormal states, and may also reveal the locations of abnormalities. The method may also be used to monitor the treatment of a subject and/or the response of disease to drugs or other treatments.

### FIELD OF INVENTION

The present disclosure relates generally to methods of Raman spectroscopy, and, in particular Raman spectral analysis for diagnostic purposes to detect and monitor potential or actual disease state of human or animal tissue.

### BACKGROUND OF THE INVENTION

Raman spectroscopy provides information about the vibrational state of molecules. Many molecules have atomic bonds capable of existing in a number of vibrational states. Such molecules are able to absorb incident radiation that matches a transition between two of the allowed vibrational states and to subsequently emit radiation. Most often, absorbed radiation is re-radiated at the same wavelength, a process designated Rayleigh or elastic scattering. In some instances, the re-radiated radiation can contain slightly more or slightly less energy than the absorbed radiation (depending on the allowed vibrational states and the initial and final vibrational states of the molecule). The energy difference between the incident and re-radiated radiation is manifested as a shift in the wavelength between the incident and re-radiated radiation. The degree of difference is designated the Raman shift (RS), measured in units of wave number (inverse length). If the incident light is substantially monochromatic (single wavelength) as it is when using a laser source, the scattered light which differs in wavelength from the incident light can be more easily distinguished from the Rayleigh scattered light.

Since Raman spectroscopy is based on irradiation of a sample and detection of scattered radiation, it can be employed non-invasively to analyze biological samples in situ. Thus, little or no sample preparation is required. In addition, water exhibits very little Raman scattering, and Raman spectroscopy techniques can be readily performed in aqueous environments.

U.S. Patent Application No. 20090040517 to Maier et al., entitled Raman Difference Spectra Based Disease Classification, discloses a method to diagnose a disease state of an unknown sample using a chemometric technique. The chemometric technique employs a principal component analysis in which a pre-determined vector space that mathematically describes the plurality of reference Raman difference data sets is selected. The test Raman data set is transformed into the pre-determined vector space. A distribution of the transformed test Raman data set in the pre-determined vector space is analyzed to generate a diagnosis.

U.S. Patent Application No. 20060281068 to Maier et al., entitled Cytological Methods for Detecting a Disease Condition such as Malignancy by Raman Spectroscopic Imaging teaches a method of assessing the disease state of mammalian cells, such as human red blood cells or human cardiac muscle cells. Raman molecular imaging (RMI) is used to detect mammalian cells of a particular phenotype. Raman scattering data relevant to the disease state of cells or tissue can be combined with visual image data to produce hybrid images which depict both a magnified view of the cellular structures and information relating to the disease state of the individual cells in the field of view. Others have performed Raman spectroscopic analysis of biological tissues. U.S. Pat. No. 6,697,665; U.S. Pat. No. 6,174,291; U.S. Pat. No. 6,095,982; U.S. Pat. No. 5,991,653; and U.S. patent application publication No. 2003/0191398. These investigators used traditional Raman sampling approaches in which tissues are analyzed by collecting a Raman spectrum from a narrowly focused point in a sample.

Descriptions of other analyses can be found in: Gelder et al., Reference Database of Raman Spectra of Biological Molecules, Journal of Raman Spectroscopy, (2007) 38: 1133-1147; Salomon et al., The Feasibility of Prostate Cancer Detection by Triple Spectroscopy, European Urology (2009) 55: 376-384; Stone et al., The Use of Raman Spectroscopy to Provide an Estimation of the Gross Biochemistries Associated with Urological Pathologies, Anal. Bioanal. Chem. (2007) 387: 1657-1668; Mahadevan-Jansen et al., Raman Spectroscopy for the Detection of Cancers and Precancers, Journal of Biomedical Optics (1996) Vol. 1, No. 1: 31-70; Gazi et al., Applications of Fourier Transform Infrared Microspectroscopy in Studies of Benign Prostate and Prostate Cancer, Journal of Pathology (2003) 201: 99-108; Crow et al., Assessment of Fiberoptic Near-Infrared Raman Spectroscopy for Diagnosis of Bladder and Prostate Cancer, Urology (2005) 65 (6): 1126-1130; and Crow et al., The Use of Raman Spectroscopy to Identify and Grade Prostatic Adenocarcinoma in vitro, British Journal of Cancer (2003) 89: 106-108.

Current diagnostic procedures for cancer and other diseases are complex, often cumbersome and usually require highly trained personnel. For most cancers, including prostate cancer, biopsies are taken from patients, which are then sent to histopathology labs where specimens are treated and observed by pathologists in an attempt to determine the presence or absence of malignancy. Because careful tissue preparation is required, this process is relatively slow and arduous. Moreover, the differentiation made by the pathologist is based on subtle morphological and other differences among normal, malignant, and benign cells. Such subtle differences can be difficult or time-consuming to detect, even for highly experienced pathologists. Accordingly, there is a need for an accurate, easier, and more efficient means of extracting sufficient information about the chemical composition of tissue to differentiate between normal and abnormal tissue for disease diagnostic purposes.

### BRIEF SUMMARY OF THE INVENTION

In accordance with an aspect of the present disclosure, there is disclosed a disease diagnostic, treatment monitoring and drug effectiveness analysis tool using Raman spectroscopy. Inelastic scattering of photons in Raman spectroscopy provides information about the chemical composition of the target molecules and enables distinction between different states of biological tissue. A general description of Raman spectroscopy has been provided above in the Background section.

In the description herein, numerous specific details are provided, such as examples of components and/or methods, to provide a thorough understanding of embodiments of the present invention. One skilled in the relevant art will recognize, however, that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the present invention.

### BRIEF DESCRIPTION OF THE SEVERAL VIEWS OF THE DRAWING

Embodiments of the present disclosure are described herein with reference to the drawings, in which:

FIG. 1 is a graphical illustration of exemplary Raman spectra of a tissue sample showing a set of selected segments for analysis, in accordance with an embodiment of the present invention;

FIG. 2 is a graphical illustration of analytical considerations of measured spectra of the first segmented region of FIG. 1, in accordance with an embodiment of the present invention;

FIG. 3 is a graphical illustration of analytical considerations of measured spectra of the second segmented region of FIG. 1, in accordance with an embodiment of the present invention;

FIG. 4 is a graphical illustration of analytical considerations of measured spectra of the third segmented region of FIG. 1, in accordance with an embodiment of the present invention;

FIG. 5 is a graphical illustration of analytical considerations of measured spectra of the fourth segmented region of FIG. 1, in accordance with an embodiment of the present invention;

FIG. 6 is a flow chart illustrating processes involved in cancer diagnostic procedures according to an embodiment of the present invention;

FIG. 7 is a flow chart illustrating processes involved in the monitoring of a patient's response to cancer treatment according to an embodiment of the present invention; and

FIG. 8 is a graphical illustration of a procedure for subtracting fluorescence from the Raman spectra according to an embodiment of the present invention.

FIG. 9 is a graphical illustration of three different exemplary Raman spectra of a normal prostate tissue sample, low-grade prostate cancer tissue sample and high-grade tissue sample showing two selected segments for analysis, in accordance with an embodiment of the present invention;

FIG. 10a is a graphical illustration of analysis of a selected segment of Raman spectra of a normal prostate tissue sample

FIG. 10b is a graphical illustration of analysis of a selected segment of Raman spectra of a low-grade prostate cancer prostate tissue sample

FIG. 10c is a graphical illustration of analysis of a selected segment of Raman spectra of a high-grade prostate tissue cancer sample

FIG. 10d is a graphical illustration of analysis of a selected segment of Raman spectra of a normal prostate tissue sample

FIG. 10e is a graphical illustration of analysis of a selected segment of Raman spectra of a low-grade prostate cancer tissue sample

FIG. 10f is a graphical illustration of analysis of a selected segment of Raman spectra of a high-grade prostate cancer tissue sample

The novel aspects of the invention as to organization and method of use, together with further objects and advantages thereof, will be better understood from the following disclosure considered in connection with the accompanying drawings in which one or more preferred embodiments of the invention are illustrated by way of example. It is to be expressly understood, however, that the detailed description and drawings are for the purpose of illustration and description only and are not intended as a definition of the limits of the invention.

As used herein, the term “comprises” refers to a part or parts of a whole, but does not exclude other parts. That is, the term “comprises” is open language that requires the presence of the recited element or structure or its equivalent, but does not exclude the presence of other elements or structures. The term “comprises” has the same meaning and is interchangeable with the terms “includes” and “has”. The term “set” has the meaning of one or more of said elements. The term “tissue” is intended to include both human and animal tissue, including individual cells, whether in situ or removed as a sample. Furthermore, any use of the term “or” as used herein is generally intended to mean “and/or” unless otherwise indicated.

### DETAILED DESCRIPTION OF THE INVENTION

In accordance with an aspect of the present disclosure, there is disclosed a diagnostic means and monitoring process that differentiates the chemical composition of diseased and normal tissue.

In accordance with another aspect of the present disclosure, there is disclosed a diagnostic means and monitoring process that utilizes a novel means for signal processing of Raman spectra to discover useful information regarding the tissue being examined.

In accordance with another aspect of the present disclosure, there is disclosed a diagnostic means and monitoring process that may be employed for early detection and treatment of diseased tissue included but not limited to cancerous tissue.

In accordance with another aspect of the present disclosure, there is disclosed a diagnostic means and monitoring process that may be used for detecting and treating various types of cancers, such as, prostate, breast cancer and cervical cancer, for example.

In accordance with another aspect of the present disclosure, there is disclosed a diagnostic means and monitoring process that may be used for detecting and treating various types of cancers or other diseases which are difficult to diagnose due to tissue characteristics (such as, for example tissue density and concentration).

The appending figures provide diagrammatic means for understanding the current invention's use of Raman spectroscopy for disease diagnosis, as well as monitoring treatment. As described in further detail below, the development of such diagnostic and monitoring methods would provide information about the state of tissue being sampled. The sampled tissue can be any tissue type, including but not limited to prostate, breast, cervical tissue, etc.

Two-dimensional maps may be subsequently generated based on histological glass slides comprised of targeted tissue. The two dimensional maps are used to extract sufficient information about the chemical composition of the tissue and to differentiate between normal and abnormal states. This information can also reveal locations of abnormalities. Comparisons of the samples of normal and abnormal tissue (for example benign hyperplasia versus adenocarcinoma) are made using baselines to determine the presence and extent of disease.

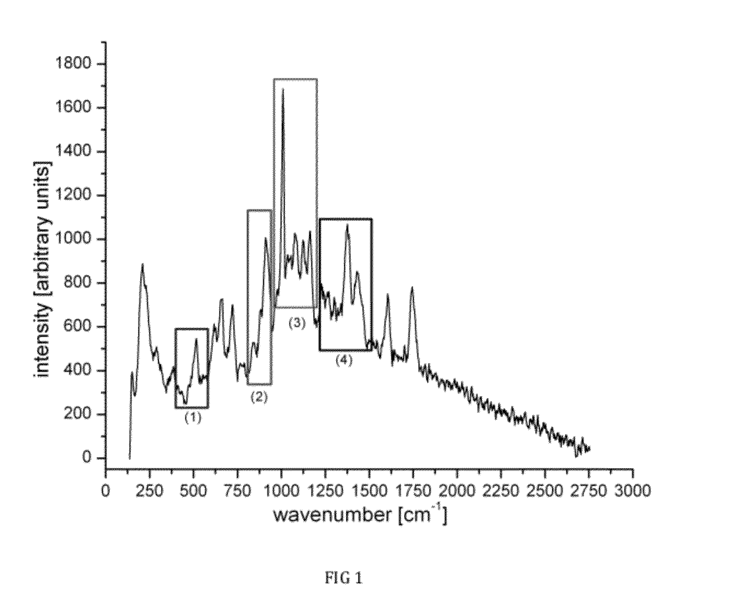

Referring to the figures, FIG. 1 is a graphical illustration of exemplary Raman spectra of a tissue sample showing a set of selected segments for analysis. The sample included a formalin fixed sample of tissue. Formalin, which is an aqueous solution of formaldehyde, is commonly used for fixing and preserving biologic specimens for pathologic and histological examination. Each histological slide was characterized via a set of measurements. The Raman peaks were decomposed and their chemical fingerprint identified. Initially, the entire spectrum was divided into segments (1), (2), (3), (4), etc., to simplify curve fitting and subsequent analysis (discussed in further detail below). The parameters extracted from the Raman bands by means of mathematical modeling were used as variables in diagnosing presence and extent of cancer. These variables were:

area under the curve (integrated intensity);

full-width-at-half-maximum;

location of a peak; and

peak height (intensity).

peak location (Raman shift)

Raman spectroscopy can be conducted utilizing various different light sources. For example, near infrared (NIR) light is used for some characterizations of biological specimens to avoid the presence of fluorescence overshadowing the Raman signal. However, in the preferred embodiment of the invention a laser in the visible wavelength range is used because it provides a stronger signal and acceptable signal-to-noise-ratio if proper focusing conditions are employed. The use of a 633 nm wavelength laser coupled with proper focusing by way of a confocal microscope provides an optimal signal-to-noise ratio for tissue samples such as prostate tissue. Other light sources and other wavelength ranges may nevertheless be employed. Most biological samples are highly fluorescent in nature. The fluorescence is treated as noise and needs to be subtracted. A number of methods to eliminate the effects of fluorescence have been developed recently. In a preferred embodiment of the invention a fitting technique proposed by Mahadevan et al. was chosen because it is relatively straightforward and yet gives very accurate results. Raw signals containing both Raman and fluorescence information are fitted with a polynomial of high enough order to describe fluorescence line shape, but not the higher frequency of a Raman line shape. The ninth order polynomial function has been found to give the best results in case of prostate samples irradiated with He—Ne laser. The fit is then subtracted from the spectrum to yield Raman signal alone, as shown in FIG. 8. The non-linear fitting process is described in detail below. To check the accuracy and quality of the pre-processing another method of fluorescence subtraction can be utilized. The same sample can be measured with two slightly different laser wavelengths. When this is done Raman bands will be different due to the difference in excitation wavelengths, but the fluorescence remains the same. The difference between the two spectra is comparable to the first derivative of the Raman spectrum. Cleaned signal can be obtained via integration of the difference between the spectra. This data can be compared to the data obtained via fitting.

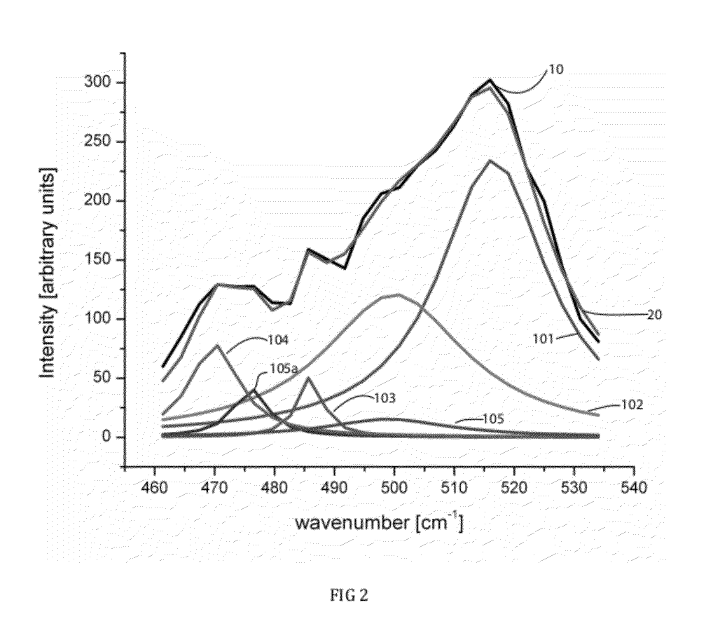

For each segment, Raman bands were decomposed and their chemical fingerprints isolated thus permitting identification of particular chemical compounds of the tissue being analyzed. This enables identification of the principal chemical compounds. The principal chemical compounds 100 are graphically illustrated in FIGS. 2-5. Variation of the principal compounds in healthy subjects are characterized and used to establish baselines.

A listing of the chemical fingerprints illustrated by the principal chemical compounds 100 of FIGS. 2-5 as follows:
101 Phosphatidylinositol
102 S-S disulfide stretching band of collagen
103 Glycogen
104 D-(+)-Mannose
105 Acetyl coenzyme A
105a Polysaccharides
106 Phosphatidic acid
107 Tryptophan
108 Formalin contamination
109 v(C—C), stretching-probably in amino acids Proline & Valine (protein band)
110 Phenylalanine
111 Collagen
112 Lipids
113 Typical phospholipids
114 v(C—C) skeletal of acyl backbone in lipid
115 Tyrosine (collagen type I)
116 T, A, G (ring breathing modes of the DNA/RNA bases)
117 CH₃ band
118 C═C stretching in quinoid ring
119 CH₂ (lipids in normal tissue)
120 CH₂ deformation 1437-42 cm⁻¹
121 Amylopectin

FIG. 2 is a graphical illustration of analytical considerations of measured spectra of the first segmented region (1) of FIG. 1. Region (1) of the measured spectra corresponds to the signal wave number range including 400-600 cm⁻¹. As shown, experimental results 10 were superimposed with curve fitted results 20. Five Raman bands 101, 102, 103, 104 and 105 were extracted in this region (1) and can be viewed as single Lorentz peaks.

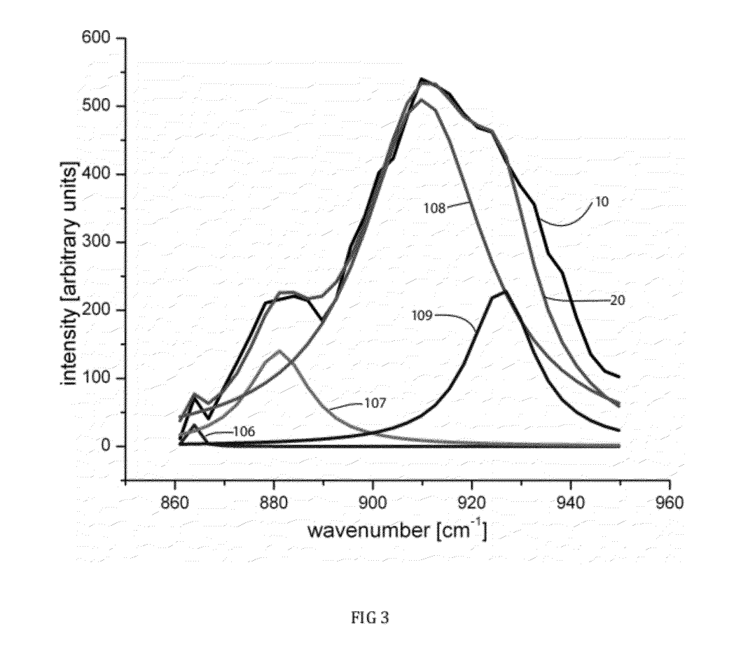

FIG. 3 is a graphical illustration of analytical considerations of measured spectra of the second segmented region (2) of FIG. 1. Region (2) corresponds to the signal wave number range including 850-950 cm⁻¹. As shown, the experimental results 10 were superimposed with curve fitted results 20. In this segment, four Raman bands 106, 107, 108, and 109 were also extracted and viewed as single Lorentz peaks.

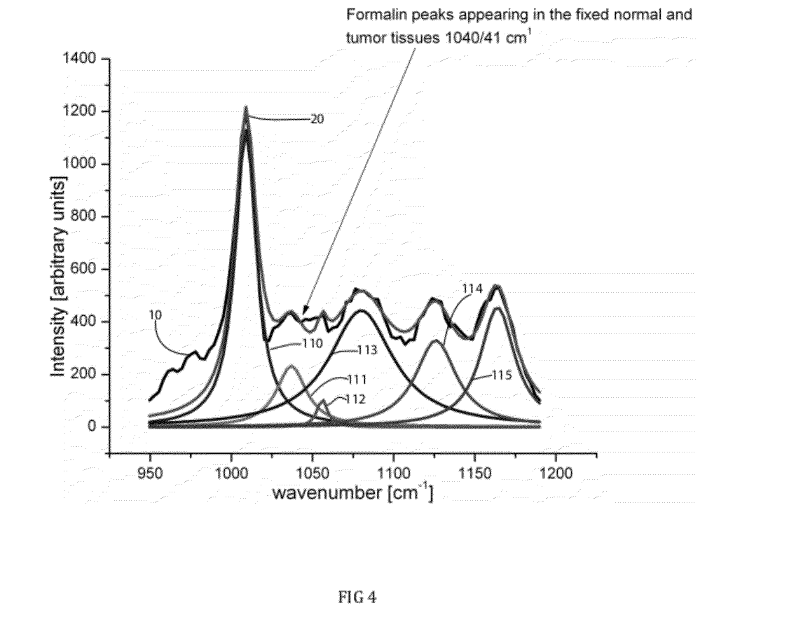

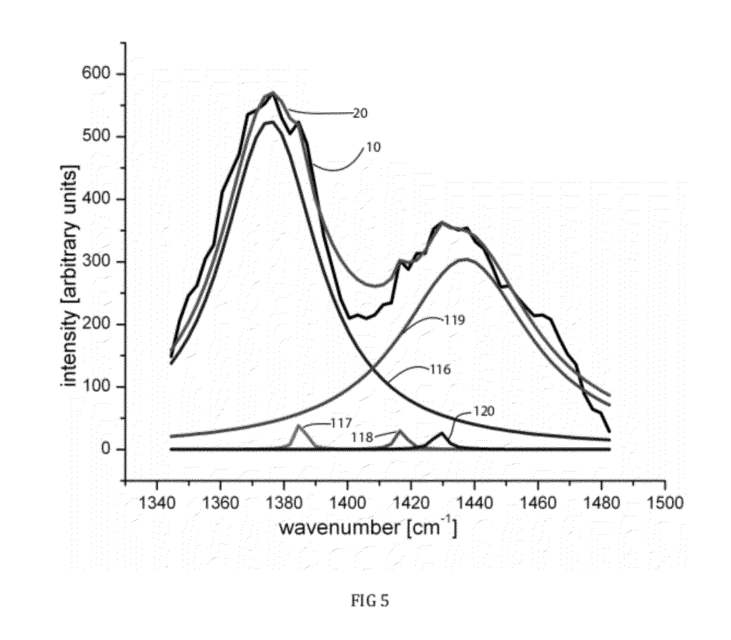

FIG. 4 is a graphical illustration of analytical considerations of measured spectra of the third segmented region (3) of FIG. 1. Region (3) corresponds to the signal wave number range including 950-1200 cm⁻¹. As shown, the experimental results 10 were superimposed with curve fitted results 20. In this segment, six Raman bands 110, 111, 112, 113, 114, and 115 were extracted and viewed as single Lorentz peaks.

Similarly as above, FIG. 5 is a graphical illustration of analytical considerations of measured spectra of the fourth segmented region (4) of FIG. 1. Region (4) corresponds to the signal wave number range including 1340-1500 cm⁻¹. As shown, the experimental results 10 were superimposed with curve fitted results 20. In this segment, five Raman bands 116, 117, 118, 119, and 120 were extracted and viewed as single Lorentz peaks.

Based on the nature of the Raman signal, the spectra were modeled as the sum of Lorentz functions with each function representing a molecular bond present in the tissue. A person of ordinary skill in the art will appreciate that curve fitting requires using a sufficient number of constraints based on the physiology of the tissue sample being analyzed to provide specific, meaningful solutions. The objective of the fitting is to mathematically describe experimental findings and thus enable detection and/or tracking of changes in chemical composition. While the preferred embodiment of the invention uses Lorentz functions as the basis for mathematical modeling, it is conceptually possible to use other mathematical approaches such as Gauss functions to perform such modeling for purpose of tissue analysis.

Because overlapping of individual Raman bands is quite common, it can sometimes be difficult to quantify the influence of particular bands on the entire spectrum. One approach is to perform a curve fit employing a function that consists of a set of individual bands. The most important aspect of curve fitting is appreciating the underlying physics behind the vibrational spectra. The nature of single band line-profiles obtained from Raman spectra can be derived either by employing hydrodynamic theory or by relating molecular vibrations to the model of a harmonic oscillator subject to an oscillating external force and friction. The latter approach is described by Marshall and Verdun and is briefly summarized below.

The line shape in vibrational spectra can be understood from the mass and spring model. Power absorption in the spectrum corresponds to the frequency of the spring and the damping coefficient can be related to the width of the spectral line of interest. If we adopt this analogy, the equation of motion can be written as:

$$m \frac{{d}^{2} x}{d {t}^{2}} = - kx - f \frac{d x}{d t} + {F}_{o} cos ω t \qquad ( 1 )$$

where −kx represents the restoring force from the spring,

$$- f \frac{d x}{d t}$$

is the friction suppressing the motion and Fₒ cos ωt is the external sinusoidally oscillating driving force. x is the steady state displacement which can be decomposed into two components x′ and x″ which represent dispersion and absorption spectra, respectively. In other words, the analogy can be explained as the weight-on-a-spring being an electron bound to an atom or molecule, and the driving force being the oscillation of the electric field of a light wave. From the equation of the motion, Eq. (1), one can obtain an expression for the absorption:

$${x}^{″} = {F}_{o} ( \frac{f ω}{{m}^{2} ( {ω}_{o}^{2} - {ω}^{2} ) + {f}^{2} {ω}^{2}} ) \qquad ( 2 )$$

where

$${ω}_{o} = \sqrt{\frac{k}{m}} .$$

From Eq. (2) it can be seen that a single band in an absorption spectra is actually of a Lorentzian profile type.

In the preferred embodiment of the invention the trust-region optimization method is employed for curve fitting of the Raman spectra. Conceptually, it is similar to the Lavenberg-Marquardt algorithm, a maximum neighborhood method developed through interpolation between the Taylor series method and the gradient descent method. Here, similarly to most least-squares estimation algorithms, it sets a problem as the iterative solving of a set of nonlinear algebraic equations. However, if the initial estimate is too far from the optimum, the algorithm will not converge. Therefore the subset of the region of the objective function is defined and optimized first. The objective is to approximate the function with a simpler one which reasonably reflects the behavior of the original function in a neighborhood E around the point x. This neighborhood referred to as the trust region. In essence, the trust region in our case represents constraints derived from the physiology of the sample tissue. For example, if it is known that phenylalanine bond is present in the prostate tissue at 1000 cm⁻¹, the model will not be permitted to vary the peak of that position more than a few wavenumbers in order to reach the optimum solution. Although trust-region optimization was employed for curve fitting in the examples described in the attached figures, it should be noted that persons skilled in the art of optimization would recognize that another optimization method could be used to achieve similar results.

One or more of the parameters described above are used to determine whether malignancy or other disease is present and the stage of malignancy or disease. The same method can be utilized for treatment monitoring. A patient undergoing treatment can be tested to see whether the molecular composition of biopsied tissue still contains traces of malignancy or disease. Doing so will give a healthcare provider information as to how effective the treatment is. The method described herein can also be used to evaluate the effectiveness of drugs or other forms of treatment. For example, some cancer drugs are designed to suppress particular metabolic processes within cancer cells. One goal in treating prostate cancer can be preventing fatty acid synthase from catalyzing fatty acids such as palmitic acid. The described method can be used to monitor changes of particular chemical components in the tissue subjected to a cancer drug and thus to determine how effective the drug is in preventing occurrence of the particular metabolic process. It is to be appreciated that one or more of the parameters described herein can be used for diagnostic purposes. Such parameters can be utilized separately or in conjunction.

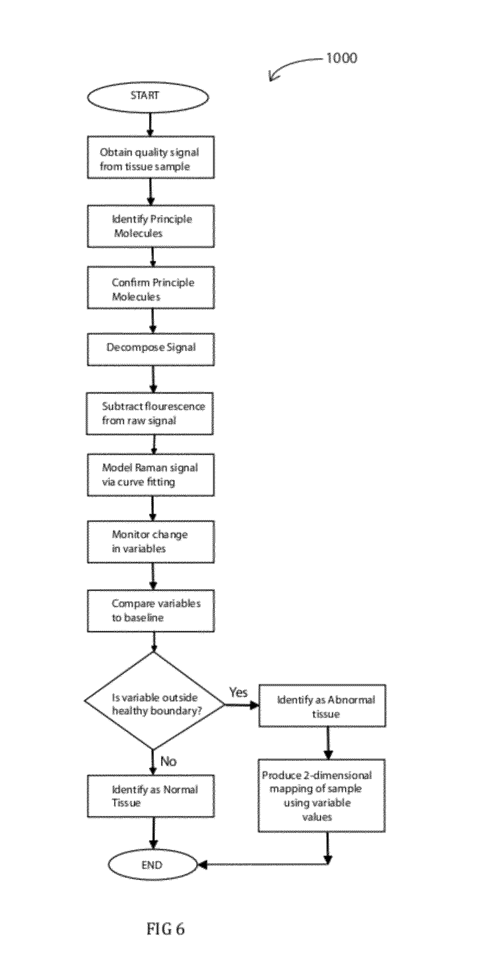

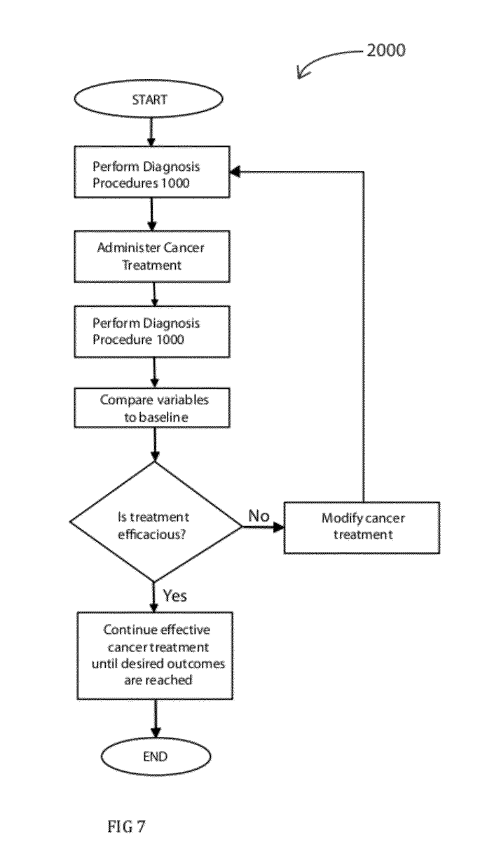

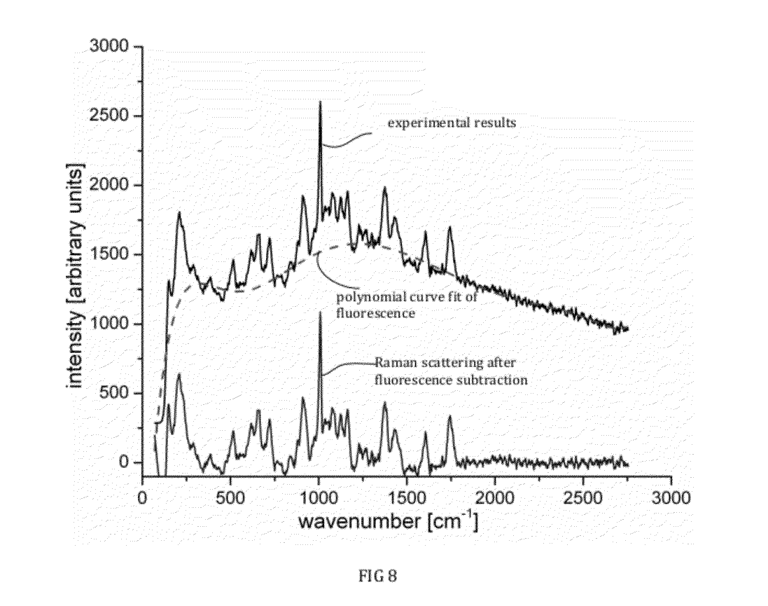

Changes in the above-defined variables are monitored as tissue is being characterized. Values of the variables are compared against pre-determined bounds set through analysis of samples of a healthy tissue. If a value for the target tissue is significantly different from the corresponding value for the reference sample, the system and methods of the instant invention suggest the presence of abnormality in the target. FIG. 6 is a flow chart illustrating a cancer diagnostic process 1000 according to an embodiment of the present invention, and as described herein above. Once a diagnosis is achieved and it is determined that a patient has diseased tissue, the instant invention further contemplates monitoring a patient's response to treatment. FIG. 7 is a diagram of a flow process of Monitoring Patient Response to Treatment 2000.

It should be appreciated that one or more of the elements depicted in the drawings/figures can also be implemented in a more separate or integrated manner, or even removed or rendered inoperable in certain cases, as may be useful in the context of a particular application. It is also within the spirit and scope of the present invention to implement a program or code that can be stored in a machine-readable medium to permit a computer to perform or assist with any of the methods and procedures described herein.

Thus, while the present invention has been described herein with reference to particular embodiments thereof, a latitude of modification, changes and substitutions are contemplated by the foregoing disclosures, It will be appreciated that in some instances some features of embodiments of the invention can be employed without corresponding use of other features and without departing from the scope and spirit of the invention as set forth. Therefore, many modifications may be made to adapt a particular situation or material to the essential scope and spirit of the present invention. It is intended that the invention not be limited to the particular terms used and/or to the particular embodiment disclosed as the best mode contemplated for carrying out this invention, but that the invention will include any and all embodiments and equivalents falling within the scope of the instant disclosure.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are included within the spirit and scope of the present invention.

### Example

The following example is provided for the purposes of illustration only and the disclosure is not limited to the specific method followed in the example.

Histopathological slides containing biopsied prostate tissue from human subjects were screened using Raman spectroscopy. Raman spectra were obtained from biopsied specimens of healthy individuals and individuals diagnosed with prostate adenocarcinoma. The samples were acquired from the Cooperative Human Tissue Network (CHTN) of the National Institutes of Health (NIH). The measurements were obtained using an assembly comprising a confocal Raman spectrometer consisting of Research Electro Optics He-NE laser emitting 632.8 nm wavelength with 30 mW maximum output power, a Nikon Eclipse TE300 inverted optical microscope, a Horiba i320 triple grating spectrometer and a deep cooled Synapse TE detector.

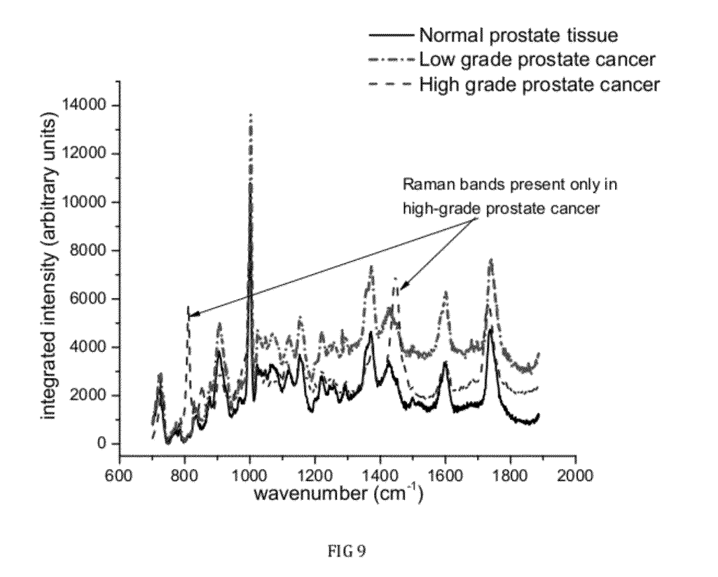

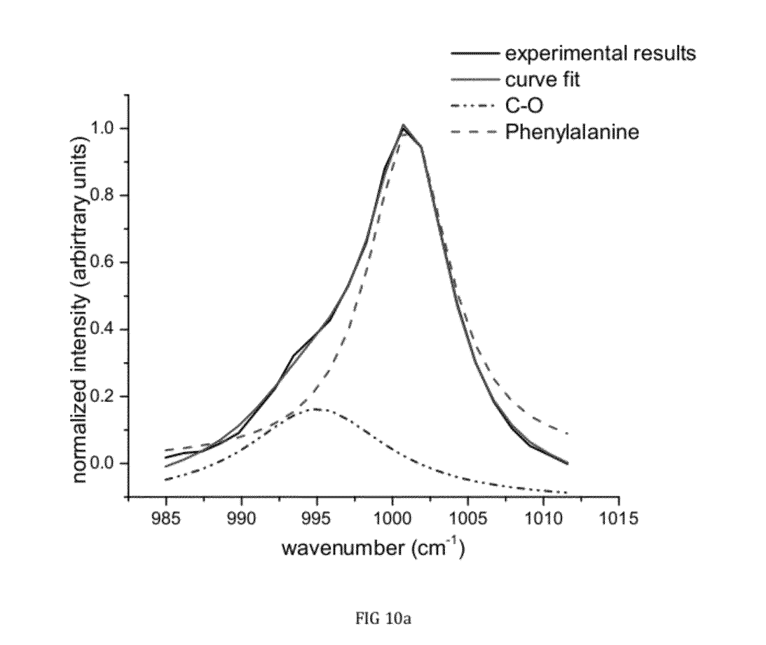

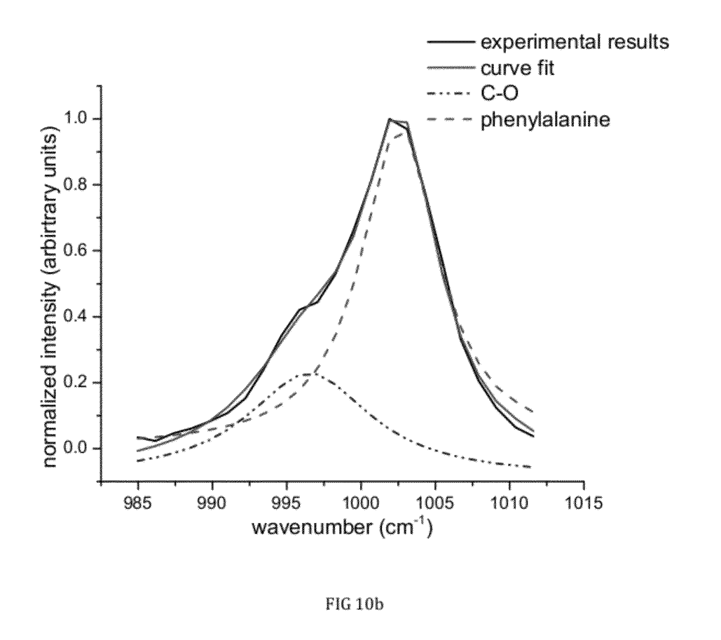

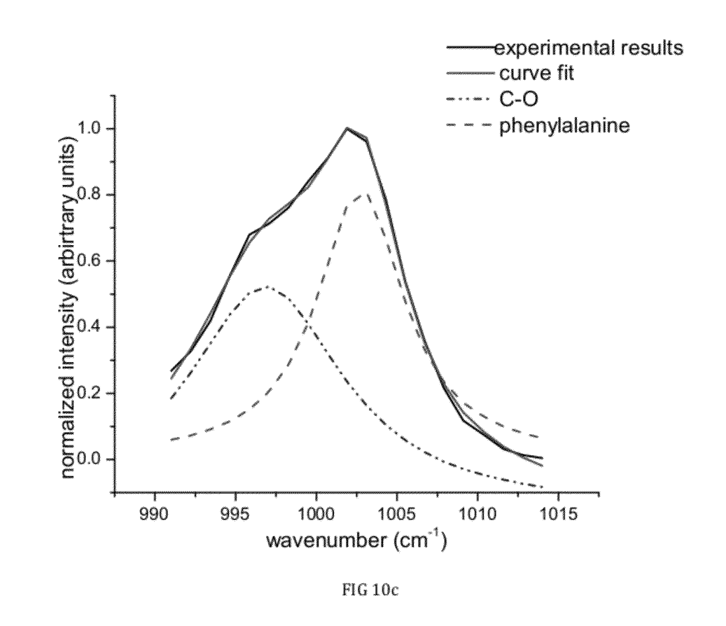

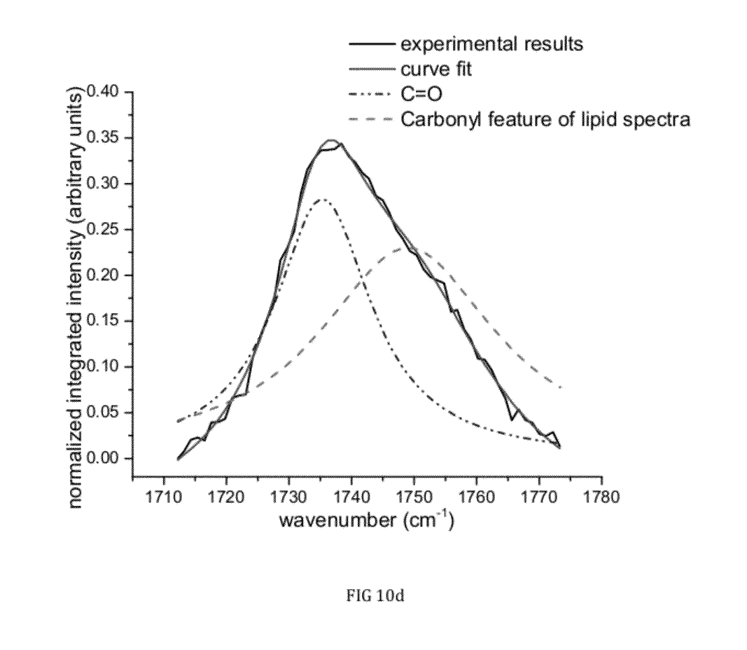

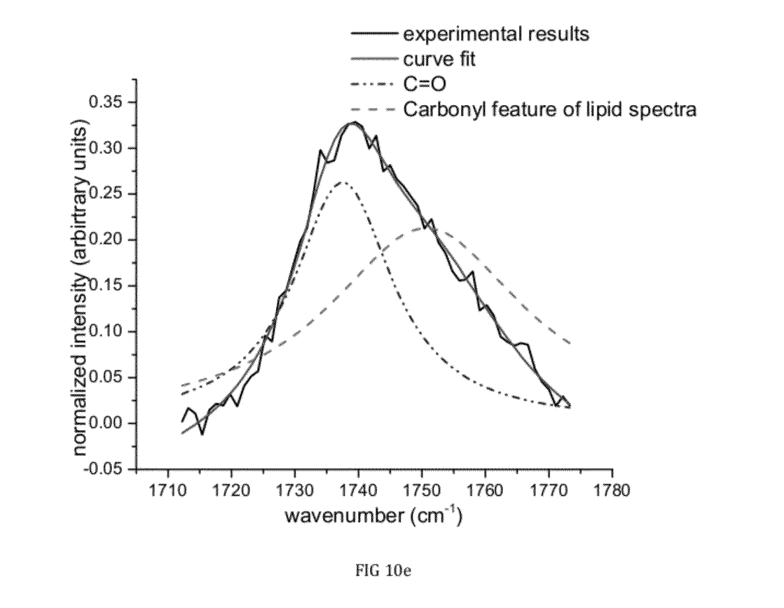

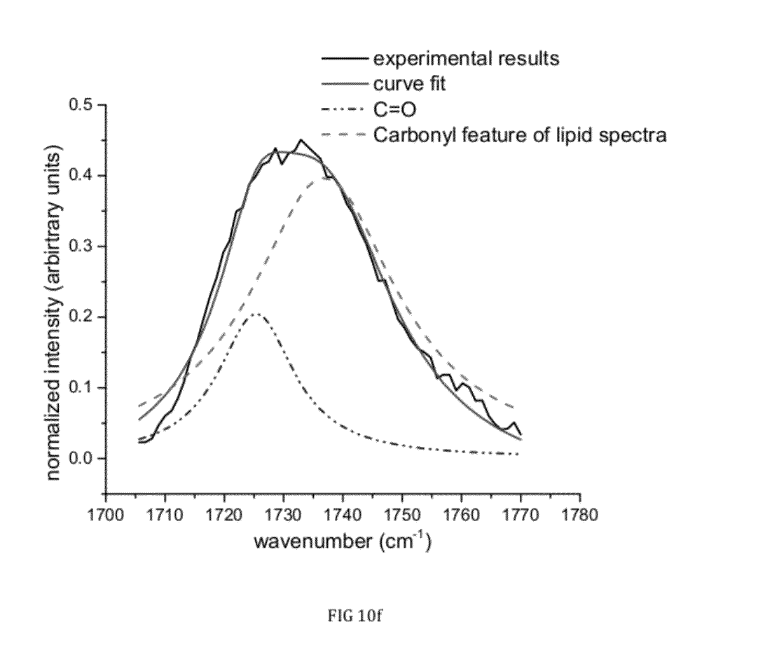

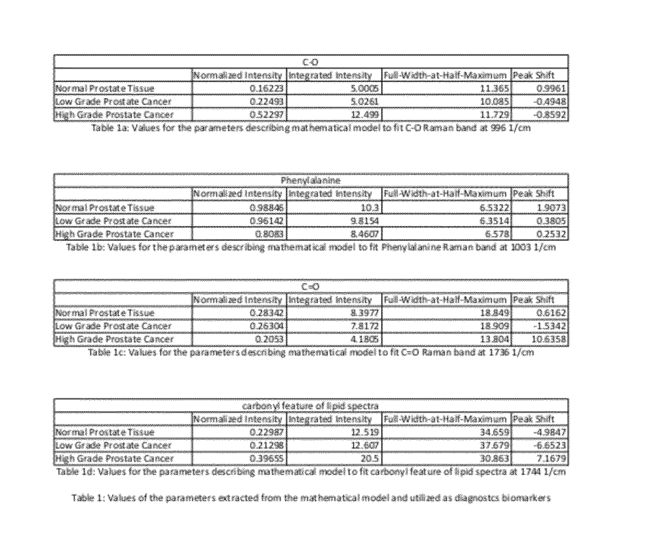

FIG. 9 shows superimposed Raman spectra of normal tissue, low grade prostate cancer and high grade prostate adenocarcinoma. Differences between Raman spectra of normal tissue and low grade prostate cancer are not immediately obvious, whereas the Raman spectra of high grade prostate cancer showed the presence of high intensity sharp peaks at 810 cm⁻¹ and 1447 cm⁻¹ which are absent in normal tissue and low grade cancer tissue. FIG. 10 show analysis of the regions of the spectra between 985 cm⁻¹ and 1011 cm⁻¹, and 1705 cm⁻¹ and 1770 cm⁻¹. Both regions of the spectra contain only two Raman bands which simplified the analysis. Intensity of the analyzed regions has been normalized with respect to the maximum value of the entire spectra. FIGS. 10a, 10b, 10c show results of the application of the analysis described herein. It can be seen that C—O located at 996 cm⁻¹ band is rather small in comparison to the phenylalanine band at 1003 cm⁻¹ in normal tissue. However the characteristics of the bands change if malignancy disease is present. In particular the peak of the C—O band shifts towards the right of the x-axis and the normalized peak height and integrated intensity increases. At the same time, the peak of the phenylalanine band shifts towards the left of the x-axis and its normalized intensity and integrated intensity decrease. The mathematical model developed is capable of quantifying these changes. The numerical values of the parameters described above are summarized in table 1.

The second analyzed region is due to the presence of a carbonyl group, which can be found in fatty acids, palmitic acid in particular. Two Raman bands are superimposed in this region, carbonyl feature of lipid spectra and C=0 band. FIGS. 10d, 10f and 10d show analysis performed on Raman spectra of normal tissue, low grade prostate cancer and high grade prostate cancer, respectively. The change in shape of the Raman spectra between 1705 cm⁻¹ and 1770 cm⁻¹ is evident. The parameter appropriately used for diagnosing malignancy is the one whose trend is consistent throughout the measurements; in this, case, integrated intensity.

The quantitative nature of the method enables a healthcare provider to utilize the method described herein to help evaluate prognosis of men with prostate cancer in a fashion similar to the Gleason grading system. In addition, in Raman spectra of highly aggressive cancers bands not seen in the spectra of normal tissue and low grade cancers are present. In FIG. 9 a phosphodiester band at 810 cm⁻¹ and CH₂ band at 1447 cm⁻¹ are identified only in high grade prostate cancer. These bands can be used as additional biomarkers to help diagnose and monitor prostate cancers.

1. A method for determining the presence of abnormalities in human or animal tissue, and/or determining the extent of abnormalities in tissue being analyzed, and/or analyzing or characterizing a subject's response to treatment and/or analyzing or characterizing the effectiveness of a drug or other disease treatment, comprising the application of Raman spectroscopy to test a tissue sample or tissue in situ, measuring Raman spectra at frequencies in the frequency shift range between 100 cm⁻¹ and 3500 cm⁻¹, processing obtained Raman spectra through decomposition of clusters of Raman bands present in a shift range between 100 cm⁻¹ and 3500 cm⁻¹ and extracting parameters from a mathematical model describing the decomposed spectra, where a significant difference between one or more of said parameters and baseline parameters derived from Raman spectroscopy of normal tissue indicates the presence of disease.

2. The method of claim 1 wherein said method involves recording a plurality of said parameters of said test sample and comparing said plurality of recorded parameters to a plurality of parameters at comparable frequencies, of said normal tissue.

3. The method of claim 1 wherein the said mathematical model comprises modeling of non-Raman features of spectra such as fluorescence and said Raman bands present in said Raman spectra.

4. The method of claim 1 wherein the said mathematical model comprises modeling said Raman bands without modeling non-Raman features such as fluorescence.

5. The method of claim 1 wherein the said mathematical model comprises modeling said Raman bands of said Raman spectra after the said spectra have been altered via mathematical or other modification.

6. The method of claim 1 wherein the method is used to identify whether cancerous tissue is from a particular stage cancer.

7. A method for determining the presence of abnormalities in human or animal tissue, and/or determining the extent of abnormalities in tissue being analyzed, and/or analyzing or characterizing a subject's response to treatment and/or analyzing or characterizing the effectiveness of a drug or other disease treatment, based on Raman spectroscopy comprising the following steps:
(i) irradiating a tissue sample or tissue in situ with light;
(ii) collecting scattered or emitted light from a tissue being analyzed wherein collected light is in the form of a wavelength resolved set of points;
(iii) selecting a vector space which mathematically describes said collected light;
(iv) analyzing said vector space;
(v) based on said analysis extracting one or more parameters such as intensity, integrated intensity, peak location (Raman shift), full-width-at-half-maximum;
(vi) comparing one or more of said parameters against one or more parameters obtained from a reference sample;
(vii) based on said analysis and comparison, describing and or classifying the target sample in terms relating to disease condition.

8. The method of claim 7, wherein said collected light includes the light emitted from or scattered by the sample and wherein said emitted or scattered light is selected from the group comprising Raman scattered light.

9. The method of claim 7 wherein the said vector space that mathematically describes said collected light comprises describing non-Raman features such as fluorescence and Raman scattered light present in said collected light.

10. The method of claim 7 wherein the said vector space that mathematically describes said collected light comprises describing said Raman scattered light without describing non-Raman features such as fluorescence.

11. The method of claim 7, wherein said parameters include but are not limited to one or more of: area under the curve (integrated intensity), full-width-at-half-maximum, location of a peak (Raman frequency shift), peak height (intensity)

12. The method of claim 7, wherein one or more of said parameters and/or a combination thereof can be used for disease detection, treatment monitoring and/or evaluation of drug effectiveness.

13. The method of claim 7, wherein said sample is fresh tissue extracted from a human or animal subject, frozen tissue, biopsy sample, a cell extracted from a subject, or a cultivated cell

14. The method of claim 7, wherein said collected light is obtained from a subject via in vivo data collection

15. The method of claim 7, wherein said reference sample is fresh tissue extracted from a subject, a frozen tissue, a biopsy sample, a cell extracted from a subject, or a cultivated cell

16. The method of claim 7, wherein the reference collected light is obtained from a subject via in vivo data collection

17. The method of claim 7, wherein the method is used to facilitate cancer prognosis diagnosis

18. The method of claim 7 wherein the method is used to identify whether the cancer is from a particular stage cancer

19. A method for determining the presence of malignancy disease in a subject, comprising obtaining Raman scattering from a test sample of tissue or individual cells from the subject, recording intensities of Raman scattering in the frequency shift range between 100 cm⁻¹ and 3500 cm⁻¹, determining from said recording the presence of the 810 cm⁻¹ Raman band at its maximum intensity and determining from said recording presence of 1447 cm⁻¹ Raman band at its maximum intensity.
